(19) United States
(12) Patent Application Publication
(10) Pub. No.: US 2005/0096524 A1
(43) Pub. Date: May 5, 2005

(54) Facemask structure improvement

(76) Inventor: Yung-Chu Cheng, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/944,731

(22) Filed: Sep. 21, 2004

(30) Foreign Application Priority Data
Nov. 5, 2003 (CN) 200320103959.5

(51) Int. Cl. A61B 5/05

(52) U.S. Cl. 600/407

(57) ABSTRACT

A facemask structure improvement including a mask body and two ear bands which can be loops or ties, with a first plurality of folds extending horizontally on the mask body, and a plurality of second folds extending vertically. Thereby, the mask can be stretched both vertically and horizontally, in a way that the mask body becomes more three-dimensional, fitting more firmly to the contours of a wearer's face, and providing more comfort and convenience than the conventional facemasks.

### BACKGROUND OF THE INVENTION

1. Field of the Invention

The present invention relates to facemasks, but more precisely, it is an improvement on existing facemask structures that renders them more three-dimensional as to fit better to the face of the wearer.

2. Description of Related Art

The major function of a facemask lies in creating a special breathing cavity, which harmful substances in the air are kept from entering that cavity, and harmful substances exhaled by the wearer are prevented from exiting that cavity. This keeps the hazardous substances in the air from being inhaled, and the exhaled hazardous substances from affecting the environment.

Different facemasks are used for different needs. Generally, the common facemasks are disposable and are usually referred to as simple facemasks. A simple facemask is one in which the filtering materials themselves are made into the face configuration, in a way that the filtering face configuration comes in direct contact with the face of the wearer. The simple facemask is well known and popular since it is cheap, easy to use, and widely available.

Referring to FIG. 1, a disposable facemask as disclosed includes a mask body 80 and two ear bands 90. The mask body 80 is made of a filtering material and is rectangular in shape, covering over the nose and mouth of the wearer. The two ear bands 90 are respectively connected to the two sides of the mask body 80 and loops around the ears of the wearer, so that the mask body 80 can be held in place to cover over the nose and mouth of the wearer.

The disposable facemask is generally made planar shaped, with ear bands 90 thereof used to loop around the ears of the wearer. Since people's faces are not planar, facemasks such as these have serious leakage problems. To resolve this problem, a plurality of folds 81 are placed on the mask body 80 of a disposable facemask extending horizontally, forming a cambered shape to cover over part of the face of the wearer.

However, the afore said conventional facemask structure has a rather cramped shape because the mask body 80 merely provides a plurality of horizontal extending folds 81 and thus the mask body 80 can only be stretched vertically. Hence, the conventional facemask cannot conform closely to the contours of the face of the wearer, allowing gaps to form causing leakages. Moreover, the conventional facemask provides a very limited cavity for the wearer to breath and converse, and comes in direct contact with mouth of the wearer when he or she tries to speak.

Therefore, due to these inconvenience and disadvantages, the conventional facemask was meant to be improved.

### SUMMARY OF THE INVENTION

The objective of the present invention is to provide a facemask which can be stretched both vertically and horizontally, thus being better three-dimensionally shaped than conventional facemask, and conforming better to the contour of the wearer's face, effectively avoiding leaks while giving the wearer the convenience of a better breathing cavity which would also make conversing easy.

To meet the objective described above, this invention provides an improvement on the existing facemask, wherein the new facemask structure includes a mask body having two pluralities of folds as well as ear bands that respectively connects to the two sides of the mask body. The two pluralities of folds consist of a first plurality of folds extending horizontally on the mask body, and a plurality of second folds extending vertically.

### BRIEF DESCRIPTION OF THE DRAWINGS

The foregoing aspects and many of the attendant advantages of this invention will become more readily appreciated as they become better understood by reference to the following detailed description, when taken in conjunction with the accompanying drawings, wherein:

FIG. 1 is a plan view showing a conventional facemask,

FIG. 2 is a perspective diagram showing a facemask structure in accordance with this invention,

FIG. 3 is a top view of a facemask in accordance with this invention, and

FIG. 4 is a schematic diagram of a facemask being worn, which is in accordance with this invention.

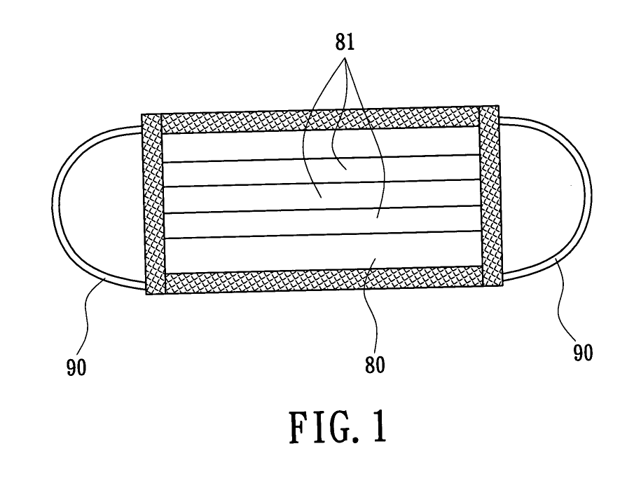

### DETAILED DESCRIPTION OF THE PREFERRED EMBODIMENTS

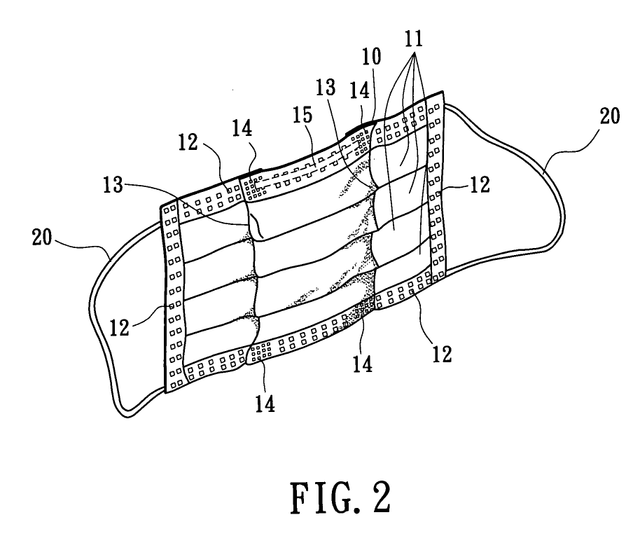

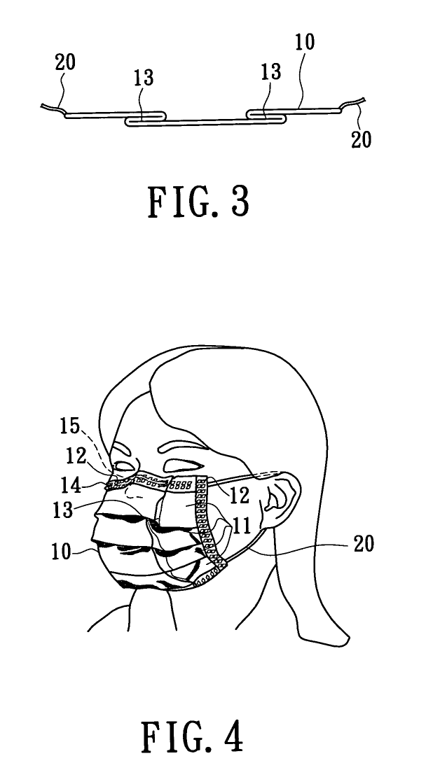

Referring to FIGS. 2 and 3, the facemask structure improvement provided by this invention includes a mask body 10 made of a multi-layered filtering material and two ear bands 20. The mask body 10 is rectangular in shape and covers the nose and mouth of the wearer.

The first plurality of folds 11 is placed extending horizontally on the mask body 10. A respective first fixing section 12 is formed by thermal pressing or stitching at the top, bottom, right and left edges of the mask body 10. The first fixing sections 12 at the right and left edges of the mask body 10 fix in place the two ends of the first plurality of folds 11 along with the two ear bands 20, such that the two ear bands 20 are respectively fixed and connected to the right and left sides of the mask body 10, thereby to be looped around the ears of the wearer when used.

Furthermore, a plurality of second folds 13 is placed on the mask body 10. In this embodiment, two of the second folds 13 are shown. The second folds 13 extend vertically on the mask body 10. A plurality of second fixing sections 14 are formed by thermal pressing or stitching at the top and bottom edges of the mask body 10, corresponding to the two ends of the second folds 13. The second fixing sections 14 are used to held fixed the two ends of the second folds 13.

In addition, a flexible nose adjustment piece 15 can be placed near the upper edge of the mask body 10 in this invention. When the facemask is worn, the nose adjustment piece 15 can be shaped to ensure that the upper edge of the mask body 10 nicely conforms to the contours of the wearer's face, such that no gap can form and the facemask remains airtight.

In this invention, by means of the first plurality of folds 11 extending horizontally on the mask body 10, the mask body 10 can be stretched vertically. By means of the second folds 13 extending vertically on the mask body 10, the mask body 10 can be stretched horizontally. Hence, the mask body 10 of this invention is more three-dimensionally shaped than conventional face masks, in a way that it fits better to the contours on the face of the wearer, as shown in FIG. 4, effectively preventing leakages, and giving the wearer greater comfort and convenience.

Moreover, since the facemask of this invention is better three-dimensionally shaped by means of a simple structural design, the cost of producing the facemask is cheaper.

The invention may be embodied in other specific forms without departing from the spirit or essential characteristics thereof. The present embodiments are therefore to be considered in all aspects as illustrative and not restrictive. The scope of this invention is indicated by the appended claims rather than by the foregoing description, and all changes that come within the meaning or the range of equivalency of the claims are therefore intended to be embraced therein.

1. A facemask structure comprising a mask body and two ear bands, with the mask body having two pluralities of folds thereon, wherein the said first plurality of folds extend horizontally on the said mask body and the said second folds extend vertically, and with the ear bands respectively connected to the right and left edges of the said mask body.

2. The facemask structure according to claim 1, wherein the said mask body further comprises a respective first fixing section formed at the top, bottom, left and right edges of said mask body, and the two ends of the said first plurality of folds are together with the two said ear bands, all fixed by the said first fixing sections at the right and left edges of the said mask body.

3. The facemask structure according to claim 1, wherein the said mask body further comprises a plurality of second fixing sections formed at the top and bottom edges, thereof corresponding to and being used to fix the two ends of the said second folds.

4. The facemask structure according to claim 1, wherein the said mask body further comprises a flexible nose adjustment piece near the upper edge of said mask body.

5. The facemask structure according to claim 1, wherein the said the ear bands are ties.
